(19) United States
(12) Patent Application Publication
(10) Pub. No.: US 2024/0242355 A1
(43) Pub. Date: Jul. 18, 2024

(54) METHOD AND DEVICE FOR AUTOMATED INSPECTION OF PLANTS AND SOLID GROWTH MEDIA

(71) Applicant: ROBOTEC PTC GMBH, Bremen (DE)

(72) Inventors: Stephan VON RUNDSTEDT, Bremen (DE); Friederike VON RUNDSTEDT, Bremen (DE)

(73) Assignee: ROBOTEC PTC GMBH, Bremen (DE)

(21) Appl. No.: 18/564,208

(22) PCT Filed: May 6, 2022
(86) PCT No.: PCT/EP2022/062274

§ 371 Date: Dec. 26, 2023

(30) Foreign Application Priority Data
May 26, 2021 (DE) 10 2021 113 510.9

(51) Int. Cl. G06T 7/00; A01G 7/00; G01N 33/00; G01N 35/00

(52) U.S. Cl. CPC G06T 7/0014; G01N 33/0098; G01N 35/0099; A01G 7/00; G06T 2207/20084; G06T 2207/30188

(57) ABSTRACT

A method and a device with which the processing of plants can be made more efficient. This is achieved by the plants and in particular plant-based and synthetic nutrient media or substrates being subjected to an automated bonitur. For this purpose, a sample and/or an image of at least one plant or of at least one nutrient medium or of the substrate is taken automatically by a sensor unit from the plant or from the nutrient medium or the substrate. This sample and/or this image are then compared by an evaluation instrument with known samples and/or recordings of plants and/or nutrient media/substrate, which have a contamination.

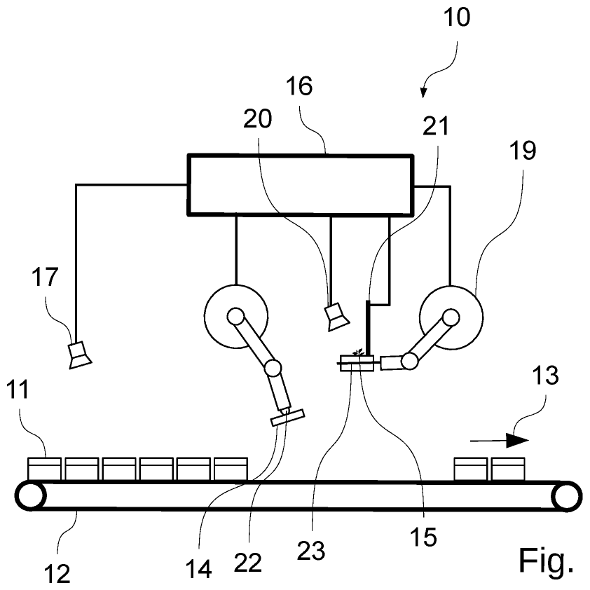

The invention relates to a method for the automated bonitur of plants and nutrient media as claimed in claim 1. The invention furthermore relates to a device for the automated bonitur of plants and nutrient media as claimed in claim 11.

For more than 40 years, plant tissue cultures have been commercially used to vegetatively propagate high-grade plant material. Large numbers of plants can in this case be produced in short periods of time with a consistent quality in sterile conditions on synthetic nutrient media in culture vessels. Other than for mass propagation, plant tissue culture is also used for further purposes in plant cultivation, for example to preserve freedom from disease and for initial propagation for simpler plant species such as bedding and balcony plants.

Contaminations in the plant tissue culture may originate from two sources, namely microorganisms on the surface or in the tissue of the plant explants or due to defective sterilization or processing methods in the laboratory. Plant surfaces and tissue are habitats for microorganisms (predominantly bacteria and fungi, but also for example mites). In the course of the plant growth and development, many microorganisms inhabiting the surface or rhizosphere may opportunistically enter the tissue of the plant through natural openings, wounds, etc., and populate the latter to a varying extent. Furthermore, facultative or obligate pathogens, either vector-assisted or by means of other penetration mechanisms, may colonize plants in a similar way or have them as host plants. Plants may therefore develop an endophytic “flora” (endogens) of variable species composition which consists of intercellular and intracellular microorganisms, including viruses, viroids, prokaryotes (bacterial and bacteria-like pathogens) and fungi. When establishing tissue cultures, depending on the explant used, superficial and endophytic microorganisms may enter the culture. In meristem culture, depending on the meristem size, most organisms are eliminated, while in the case of leaf, petiole or stem explants, most if not all microorganisms may be transferred into the tissues.

For economically viable in vitro propagation in a short time with high volumes, it is essential to keep the in vitro cultures in an equilibrium with these microorganisms, so that proliferation of for example bacteria or fungi does not occur on the nutrient medium. Each processing or propagation step is carried out under sterile conditions on the sterile workbench, since otherwise the risk of contaminating the in vitro cultures with exogenous microorganisms occurs. After the subculturing period of the plants to be propagated on the synthetic nutrient medium under artificial conditions, the plants are reprocessed. Before this reprocessing, the plants, or the culture vessels, are subjected to visual inspection (bonitur) for contaminations and quality so that no contaminated and/or qualitatively deficient plant cultures reach propagation. For this bonitur, the plants are examined individually by persons. This makes the method not only very time-consuming but also cost-intensive.

The object of the invention is to provide a method and a device with which the processing of plants can be made more efficient.

A solution to this object is described by the measures of claim 1. Accordingly, the plants and in particular plant-based and synthetic nutrient media or substrates are subjected to an automated bonitur. For this purpose, a sample and/or an image of at least one plant or of at least one nutrient medium or of the substrate is taken automatically by a sensor unit from the plant or from the nutrient medium or the substrate. This sample and/or this image are then compared by an evaluation instrument with known samples and/or recordings of plants and/or nutrient media/substrate, which have a contamination. By this automated comparison of the plants, or of the nutrient media or of the substrate, with samples or recordings which have a contamination or the like, a visual inspection may be carried out rapidly and reliably. The time-consuming and cost-intensive use of persons as well as erroneous visual measurements and counting may therefore be avoided. The method described here is used, in particular, for the processing of crop plants and ornamental plants. It is, however, likewise conceivable for the claimed method to be used for the bonitur of other subjects as well. The claimed plants may also be plant organ parts or other plant material, tissue, cells or the like. Accordingly, the method described here is not intended to be restricted to use on a plant, but may instead also extend to the use of other organic forms. As an alternative to the nutrient medium claimed here, the method and the device may also be used for container contents or substrates.

In particular, the invention furthermore proposes that the at least one plant, in particular cells, tissue, plant organs, and/or the at least one nutrient medium or the substrate, is delivered to the sensor unit in at least one container or on a tray before the bonitur, the delivery being carried out manually by a person or automatically by a conveyor or a gripper arm, the container being delivered accurately by the gripper arm to the sensor unit in such a way that the sample and/or the image can be taken in a particularly efficient way. In the case of manual delivery of the container to the sensor unit, the person holds the container under the sensor unit for a sufficiently long period of time. The person may in this case be informed when the sample or the image has been taken and how much of a sample or plant or the like has been rejected. Further, the quality defect that has been detected is documented. In the case of automatic delivery, no further use of persons is necessary. In this case, the delivery and removal of the containers are carried out and monitored by a control unit. It is moreover conceivable that the at least one plant, in particular cells, tissue, plant organs, and/or the at least one nutrient medium or the substrate, is deposited on a rack or a tray and delivered to the sensor unit by the gripper arm before the bonitur, or that the sensor unit is delivered to the rack.

Preferentially, an image recognition instrument furthermore takes an image of a container or of a multiplicity of containers, each of which has a plant and/or a nutrient medium, and with the aid of this image the gripper arm is automatically brought to a container in order to grip the container and deliver it to the sensor unit. The image recognition instrument therefore recognizes individual containers from the multiplicity of containers and can then particularly efficiently grip them successively. Alternatively, it is also conceivable that a plurality of containers are simultaneously gripped and delivered to the sensor unit, or that the sensor unit is delivered to the containers and scans the individual containers.

Preferably, it is also conceivable that the individual containers are transported on a conveyor through a first airlock into a room, in particular a sterile room, the bonitur being carried out before entry into the first airlock or not until inside the room. After the processing in the room, the individual containers can then be transported away through a second airlock out of the room. The airlock can prevent any contaminations or the like from being carried into the sterile interior. This delivery and withdrawal of the individual containers may take place both manually and in an automated fashion.

It is furthermore conceivable that the individual containers are transported into the room in a closed state and then opened manually or by a gripper arm and the bonitur is then carried out in the room. In order to open the containers, it is for example possible to use a robot arm having a suction cup, which lifts the lid of the container and deposits it back onto the container after the processing. During the processing, the lid may be lifted in a magazine for a plurality of lids, each lid in turn being reassigned precisely to the same container. Alternatively, it is also conceivable that new, in particular sterile, lids from a lid dispenser are used in order to reclose the container.

According to a further particularly advantageous exemplary embodiment of the invention, the sample and/or the recording is taken by at least one camera for visible, infrared and/or ultraviolet light, a pH measuring instrument, an impedance spectroscope, a gas sensor or the like. A multiplicity of measurements, in particular complementary measurements, may be carried out by these sensors in order to ascertain any contaminations or infections of the plant or of the nutrient medium or of the substrate.

Further, according to the invention an evaluation of the samples and/or images taken may be carried out by a neural network, the neural network or the evaluation instrument being provided with a database having a multiplicity of samples and/or images which can be correlated with a contamination or an infection of the plant or of the nutrient medium/substrate. By the use of a neural network, the evaluation instrument can progressively learn to do this and improve the recognition of contaminations. By the database provided, the recognition of contaminations or infections is carried out very rapidly and reliably. This database may be extended arbitrarily for each new type of plant or nutrient medium.

According to an additional exemplary embodiment of the invention, a plurality of the aforementioned sensor instruments may be used simultaneously or successively for carrying out the bonitur, in particular with the neural network ascertaining while the sample and/or the image is being taken whether a further sample and/or a further image is to be taken. Particularly in cases in which the result is not unequivocal or needs to be verified, it may be advantageous that a further measurement is carried out in order to confirm the measurement result. By this possibility of multiple use of the sensors, the reject rate of unrecognized samples or images may be reduced. Lastly, it is also conceivable that in equivocal or doubtful cases individual plants, substrates, or pots or trays are additionally checked by a person in order to reintroduce them into the further processing chain or entirely reject them, as appropriate.

Furthermore, a further aspect of the invention may consist in the neural network or the artificial intelligence ascertaining on the basis of the comparison of the samples and/or images taken of the plant or of the nutrient medium or of a substrate with the samples and/or images stored in the database whether the plant or the nutrient medium, or the substrate, is delivered to further method steps, rejected or subjected to a special treatment. By this further aspect, the method described here consists not only in carrying out a visual inspection of the plant, or of the nutrient medium/substrate, but at the same time also in a more far-reaching decision about the way in which the plant or the nutrient medium or the substrate should be dealt with after the inspection. With this further method step, the processing may be controlled by objective criteria. While the person has previously had to decide how the plant or the nutrient medium, or the substrate, should be dealt with further, this is now done uniformly on the basis of the neural network.

Lastly, according to the invention it is conceivable that in the event of a contamination or an infection of the plant or of the nutrient medium/substrate being ascertained, the evaluation instrument generates a corresponding signal or rejects the container, preferably by using a gripper arm. By the deliberate rejection of the container, the ascertained contamination is eliminated very effectively from the process, so that the other plants or nutrient media are correspondingly protected. Early recognition of a contamination can protect a multiplicity of further plants and nutrient media, so that the overall process may be made more cost-efficient.

A device for achieving the object mentioned in the introduction is described by claim 11. Accordingly, the device for the automated bonitur of plants and in particular plant-based or synthetic nutrient media has a sensor unit for the automatic taking of a sample and/or an image of at least one plant or of at least one nutrient medium/substrate. The device furthermore has an evaluation instrument for the automated recognition of a contamination of the plant or the nutrient medium/substrate.

The sensor unit may be a camera which is sensitive to visible, infrared and/or ultraviolet light. While some infections or fungi can be recognized directly, other contaminations may become visible in a different wavelength range. In particular by illuminating the plant or the nutrient medium/substrate with light of a particular wavelength, spores or the like, which would not be recognizable in the visible spectrum, may be made visible by fluorescence. In order to make such contaminations recognizable, hyperspectral cameras in particular are suitable. In order to recognize in particular very small fungi, bacterial colonies, mites, etc., it is also conceivable to use a magnifying lens or a microscope with an imaging sensor unit. The sensor unit may moreover be configured as a pH measuring instrument. Infections of the medium may be ascertained particularly early and accurately through the variation of the pH. For this purpose, a corresponding sample may be removed from the plant or the nutrient medium and thereupon be measured by the measuring instrument. It is furthermore conceivable that further properties of the plant or of the nutrient medium, which indicate a possible contamination or infection, are determined by an impedance spectroscope. According to a further embodiment, the sensor unit may be configured as a gas sensor. By determining the composition of the atmosphere and optionally a variation of the composition, infections may be ascertained even if they are not yet visible. According to one particularly advantageous embodiment, the device may also have a plurality of the aforementioned sensor units in order to perform different measurements on the plant or the nutrient medium. The measurement result may be improved by a supplementary measurement.

Furthermore, according to the invention, it is conceivable that the evaluation instrument is based on an artificial intelligence having a neural network, the neural network having a database in which a multiplicity of samples and/or images of plants and nutrient media having a contamination or an infection are stored. By this artificial intelligence, patterns as well as features of the individual images and samples are recognized and compared with known data. The samples and/or images taken in this case are used to increase the database and therefore constantly improve the accuracy of the neural network.

Lastly, the device may have at least one conveyor instrument or gripper arm, in particular a robot arm, by which a container, in which the plant and/or the nutrient medium are placed, can be delivered to the sensor unit. This conveyor instrument, or the gripper arm, is controlled by a control instrument which is in communication with the neural network and is controlled on the basis of the sensor unit or image recognition instrument. By this combination, or integration, of various constituent parts of the device, the prescribed method may be carried out in a particularly time-efficient and cost-efficient fashion.

A possible exemplary embodiment of the invention will be described in more detail below with the aid of the single FIGURE of the drawing.

FIGURE a representation of a highly schematized device.

The FIGURE represents a possible exemplary embodiment of the device 10 according to the invention. According to the invention, this device 10 may also be integrated into other devices (not represented here) and may constitute a part of a complex plant processing operation.

In the exemplary embodiment represented in the FIGURE, a plurality of containers 11 are transported in a conveyor direction 13 on a conveyor instrument 12. These containers 11 can be closed by a lid 14. The containers 11 contain both a nutrient medium (not visible) or substrate and a plant 15. The individual containers 11 can be deposited manually or in an automated fashion onto the conveyor instrument 12. The containers 11 may in this case already have passed through an airlock (not represented), or they are delivered in the further course of the method to an airlock in order to be processed further in a sterile room.

The device 10 moreover comprises a control unit 16. In the exemplary embodiment represented here, this control unit 16 controls an image recognition instrument 17, a first gripper arm 18, a second gripper arm 19, a first sensor unit 20 and a second sensor unit 21. The conveyor instrument 12 may moreover be controlled via the control unit 16. In order to control the aforementioned components, the control unit 16 comprises at least a processor and an evaluation instrument. The control unit 16 may furthermore also have a neural network which assists the method described here.

First, the containers 11 transported on the conveyor instrument 12 are registered by the image recognition instrument 17. The image recognition instrument 17, or the control unit 16, determines the nature and/or size of the individual containers 11 and the number of individual containers, and optionally reads identification numbers or descriptions on the individual containers 11. With the aid of these information items, the control unit 16 ascertains the next process step. In the case of a container 11 closed by a lid 14, this lid 14 is first lifted by the first gripper arm 18. For the further course of the method, this lid 14 may either remain on the gripper arm 18 or be delivered to a magazine (not represented). During the subsequent further transport of the container 11, the same lid 14 is preferably returned to the same container 11. In order to handle the lid 14, the gripper arm 18 has a corresponding gripping means 22. This gripping means 22 may, for example, be a suction cup. This gripping means 22 is arranged movably on the robot arm-like gripping arm 18 and is likewise controlled by the control unit 16. Preferably, for this purpose the gripper arm 18 is movable in three-dimensional space.

In a subsequent method step, the opened container 11 with the nutrient medium/substrate and/or the plant 15 is delivered to a sensor unit. In the exemplary embodiment represented here, the container 11 is delivered to two sensor units 20, 21. This delivery is carried out by the second gripper arm 19. This second gripper arm 19 is likewise configured in the manner of a robot arm and also has a gripping means 23. This gripping means 23 is configured in such a way that it can grip the container 11 and deliver it accurately to a sensor unit. The container is then deposited back on the conveyor instrument 12 by the gripper arm 19 and closed with the lid 14.

The sensor units 20, 21 represented in the FIGURE comprise a camera which can record an image, or a recording, of the plant 15 or of the nutrient medium. The second sensor unit 21 is a measuring instrument for determining the pH of the nutrient medium. It is however likewise conceivable for the device 10 to have only one sensor unit or further sensor units. By these supplementary measurements, the bonitur both of the plant 15 and of the nutrient medium may be carried out.

The information items recorded by the sensor units 20, 21 are evaluated by the control unit 16. The neural network may in this case be employed, the neural network comparing the recorded information items, or the recordings and the samples, with stored patterns. The stored patterns may, for example, represent recordings or samples of corresponding plants or nutrient media which have a contamination or an infection. The control unit can carry out the bonitur by this comparison of the known data with the data that have been taken by the sensor units 20, 21. The further treatment of the plant 15, or of the nutrient medium, is carried out as a function of the result of the bonitur.

Because of the constant pattern recognition by the neural network, the network is continuously trained so that the probability of success is improved with each comparison carried out. By the use of the neural network, a bonitur may also be carried out flexibly on different plants, or different nutrient media, since the neural network recognizes which type of plant or nutrient medium is involved. The neural network may resort to different data sets as a function of the plant or nutrient medium recognized.

As an alternative to the exemplary embodiment represented in the FIGURE, it is likewise conceivable that a person opens the containers 11, delivers them to the sensor unit 20, 21 and then, for example, deposits them on a conveyor (not represented). It is also conceivable that a person assists the method represented here by the person confirming or denying the contamination recognized by the neural network, or the artificial intelligence, in order to train the neural network even further.

A further aspect of the invention might consist in the control unit 16 operating further instruments which thereupon treat the plant 15 or the nutrient medium, the substrate, the container and/or the tray after an infection has been ascertained. For example, the opened container 11 might be exposed to a short burst of UV, UVC or X-radiation, chemical spraying or gassing with H₂O₂, chlorine dioxide, NaOCI, a surface disinfectant, or the like, in order to kill ascertained germs, etc. It is also conceivable to re-sterilize plants, substrates or pots by means of a CO₂ pressure if an animal contamination is ascertained. If necessary, the container 11 might also thereupon be delivered to a conveyor (not represented) for rejection of the plant 15 or of the nutrient medium.

### LIST OF REFERENCE SIGNS

10 device
11 container
12 conveyor instrument
13 conveyor direction
14 lid
15 plant
16 control unit
17 image recognition instrument
18 first gripper arm
19 second gripper arm
20 first sensor unit
21 second sensor unit
22 gripping means
23 gripping means

1. A method for the automated bonitur of plants, wherein an image and/or a sample of at least one plant or of at least one nutrient medium or of a substrate is taken automatically by a sensor unit and this sample and/or this image are compared by an evaluation instrument with known samples and/or recordings of plants and/or nutrient media or substrates, which have a contamination.

2. The method as claimed in claim 1, wherein the at least one plant and/or the at least one nutrient medium or the substrate is delivered to the sensor unit in at least one container or on a tray, the delivery being carried out manually by a person or automatically by a conveyor or a gripper arm, arm, the container or the tray being delivered accurately by the gripper arm to the sensor unit in such a way that the sample and/or the image can be taken in a particularly efficient way.

3. The method as claimed in claim 1, wherein an image recognition instrument takes an image of a container or of a multiplicity of containers, each of which has a plant and/or a nutrient medium or a substrate, and with the aid of this image the gripper arm is automatically brought to a container in order to grip the container and deliver it to the unit.

4. The method as claimed in claim 1, wherein the individual containers are transported on a conveyor instrument through a first airlock into a room, in particular a sterile room, the bonitur being carried out before entry into the first airlock or in the room.

5. The method as claimed in claim 4, wherein the individual containers are transported into the room in a closed state and then opened manually or by a gripper arm and the bonitur is then carried out in the room.

6. The method as claimed in claim 1, wherein the sample and/or the recording is taken by at least one camera for visible, infrared and/or ultraviolet light.

7. The method as claimed in claim 1, wherein an evaluation of the samples and/or images taken is carried out by an artificial intelligence (AI), the AI or the evaluation instrument being provided with a database having a multiplicity of samples and/or images which can be correlated with a contamination or an infection of the plant or of the nutrient medium or of the substrate.

8. The method as claimed in claim 1, wherein a plurality of the aforementioned sensor instruments are used simultaneously or successively for carrying out the bonitur.

9. The method as claimed in claim 1, wherein on the basis of the comparison of the samples and/or images taken of the plant or of the nutrient medium or of the substrate with the samples and/or images stored in the database, the neural network ascertains whether the plant the nutrient medium is delivered to further method steps, rejected or subjected to a special treatment.

10. The method as claimed in claim 1, wherein in the event of a contamination or an infection of the plant or of the nutrient medium or of the substrate being ascertained, the evaluation instrument generates a corresponding signal or rejects the container.

11. A device for the automated bonitur of plants, having a sensor unit for the automated taking of an image and/or a sample of at least one plant or of at least one nutrient medium or of a substrate and an evaluation instrument for the automated recognition of a contamination on the plant or the nutrient medium or the substrate.

12. The device as claimed in claim 11, wherein the sensor unit is at least one camera, for visible, infrared and/or ultraviolet light, a pH measuring instrument, an impedance spectroscope, a gas sensor or the like.

13. The device as claimed in claim 11, wherein the evaluation instrument is based on an artificial intelligence having a neural network, the neural network having a database in which a multiplicity of samples and/or images of plants or nutrient media or substrates having a contamination or an infection are stored.

14. The device as claimed in claim 11, wherein the device has at least one conveyor instrument or gripper arm, by which a container or a tray, in or on which the plant and/or the nutrient medium are placed, can be delivered to the sensor unit.
